(19) United States
(12) Patent Application Publication
(10) Pub. No.: US 2022/0170094 A1
(43) Pub. Date: Jun. 2, 2022

(54) SINGLE TUBE PREPARATION OF DNA AND RNA FOR SEQUENCING

(71) Applicant: Roche Sequencing Solutions, Inc., Pleasanton, CA (US)

(72) Inventors: Daniel M. Klass, Livermore, CA (US); Alexander Lovejoy, Newark, CA (US); Melissa Loyzer, Hamilton (CA); Kelly M. McClintock, Mountain View, CA (US)

(21) Appl. No.: 17/651,135

(22) Filed: Feb. 15, 2022

Related U.S. Application Data
(63) Continuation of application No. PCT/EP2020/072958, filed on Aug. 17, 2020.
(60) Provisional application No. 62/888,963, filed on Aug. 19, 2019.

(51) Int. Cl. C12Q 1/6874; C12Q 1/6855; C12Q 1/6806; C12N 15/10

(52) U.S. Cl. CPC C12Q 1/6874; C12N 15/1093; C12Q 1/6806; C12Q 1/6855

(57) ABSTRACT

The present invention is a method and compositions for forming a library for nucleic acids sequencing simultaneously from DNA and RNA present in a sample.

### CROSS-REFERENCE TO RELATED PATENT APPLICATIONS

This application is a continuation of PCT Application No. PCT/EP2020/072958, filed on Aug. 17, 2020, which claims the benefit of U.S. Provisional Application Ser. No. 62/888,963, filed on Aug. 19, 2019, the entireties of each which are incorporated herein by this reference.

### FIELD OF THE INVENTION

The invention relates to the field of nucleic acid sequencing. More specifically, the invention relates to the field of enriching rare nucleic acid targets for sequencing.

### BACKGROUND OF THE INVENTION

Many workflows exist for the separate preparation and sequencing of DNA samples and RNA samples. When information is desired from both DNA and RNA, some workflows are completely separate while others may join as one workflow at some step along the way. In either case, if information is desired from both DNA and RNA from a single source (e.g. patient sample), two separate specimens are required—one to isolate DNA, and a second to isolate RNA. This results in a waste of sample material as well as labor and reagent costs. For precious samples, such as clinical biopsy samples, forensic or historical, there may not be enough material to perform two separate isolations for DNA and RNA. A combined DNA and RNA workflow is considered impractical for the fear that RNA-related steps may harm the DNA (e.g. through denaturation) while DNA-related steps may be damaging to RNA. There is a need for a robust combined RNA/DNA workflow that would reliably recover DNA and RNA from a sample in the same tube.

### SUMMARY OF THE INVENTION

The invention is a method of forming a DNA library suitable for nucleic acid sequencing or other downstream analysis wherein the target sequences in the library originate from both cellular RNA and cellular DNA. While DNA and RNA targets are processed simultaneously in a single workflow, they remain distinguishable as originating from DNA or RNA respectively after completion of the method.

In some embodiments, the invention is a method of preparing a mixture of RNA and DNA targets for sequencing the method comprising: providing a sample comprising RNA and DNA targets; contacting the sample with a target-specific primer under conditions that do not allow DNA denaturation; extending the target-specific primer hybridized to at least one RNA target with a nucleic acid polymerase having reverse transcriptase activity to form a cDNA strand; and contacting the sample with an RNaseH activity and a nucleic acid end repair activity to form a mixture of double-stranded DNA and double-stranded cDNA. The target-specific primer may comprise a barcode that distinguishes cDNA molecules from DNA molecules in the mixture of double-stranded DNA and double-stranded cDNA. The nucleic acid end repair activity may consist of a mixture of a DNA polymerase, an exonuclease and a polynucleotide kinase. In some embodiments, the method further comprising a preliminary step of fragmenting the RNA and DNA targets. In some embodiments, the method further comprising contacting the mixture of double-stranded DNA and double-stranded cDNA with an adaptor having one or more barcodes, e.g., a unique molecular identifier (UID) and a sample identifier (SID). In some embodiments, the method further comprising a step of sequencing the adapted DNA optionally, after amplifying the adapted DNA prior to sequencing.

In some embodiments, the invention is a library of nucleic acids formed by a method comprising providing a sample comprising RNA and DNA targets; contacting the sample with a target-specific primer under conditions that do not allow DNA denaturation; extending the target-specific primer hybridized to at least one RNA target with a nucleic acid polymerase having reverse transcriptase activity to form a cDNA strand; and contacting the sample with an RNaseH activity and a nucleic acid end repair activity to form a mixture of double-stranded DNA and double-stranded cDNA. The mixture of double-stranded DNA and double-stranded cDNA may further comprise adaptors. The target-specific primer may comprise a barcode so that the double-stranded cDNA is distinguishable from the double-stranded DNA by the presence of the barcode.

In some embodiments, the invention is a kit for preparing a mixture of RNA and DNA targets for sequencing by the novel method described herein, the kit comprising: one or more target-specific primers having a barcode; a nucleic acid polymerase having reverse transcriptase activity; an RNaseH; a DNA polymerase having a 3′-5-exonuclease activity; a polynucleotide kinase and optionally, an adaptor and a DNA ligase, wherein the adaptor comprises one or more molecular barcodes and a universal primer binding site.

### BRIEF DESCRIPTION OF THE DRAWINGS

FIG. 1 is a diagram of the initial part of the workflow.

FIG. 2 is a diagram of a part of the workflow.

FIG. 3 is a diagram of the final part of the workflow.

FIG. 4 shows experimental validation of the combined DNA/RNA protocol applied to a cell line sample.

### DETAILED DESCRIPTION OF THE INVENTION

### Definitions

Unless defined otherwise, technical and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012).

The following definitions are provided to facilitate understanding of the present disclosure.

The term “barcode” refers to a nucleic acid sequence that can be detected and identified. Barcodes can generally be 2 or more and up to about 50 nucleotides long. Barcodes are designed to have at least a minimum number of differences from other barcodes in a population. Barcodes can be unique to each molecule in a sample or unique to the sample and be shared by multiple molecules in the sample. The term “multiplex identifier,” “MID” or “sample barcode” refer to a barcode that identifies a sample or a source of the sample. As such, all or substantially all, MID barcoded polynucleotides from a single source or sample will share an MID of the same sequence; while all, or substantially all (e.g., at least 90% or 99%), MID barcoded polynucleotides from different sources or samples will have a different MID barcode sequence. Polynucleotides from different sources having different MIDs can be mixed and sequenced in parallel while maintaining the sample information encoded in the MID barcode. The term “unique molecular identifier” or “UID,” refer to a barcode that identifies a polynucleotide to which it is attached. Typically, all, or substantially all (e.g., at least 90% or 99%), UID barcodes in a mixture of UID barcoded polynucleotides are unique.

The term “DNA polymerase” refers to an enzyme that performs template-directed synthesis of polynucleotides from deoxyribonucleotides. DNA polymerases include prokaryotic Pol I, Pol II, Pol III, Pol IV and Pol V, eukaryotic DNA polymerase, archaeal DNA polymerase, telomerase and reverse transcriptase. The term “thermostable polymerase,” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. In some embodiments, the following thermostable polymerases can be used: Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulforococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases. In some cases, the nucleic acid (e.g., DNA or RNA) polymerase may be a modified naturally occurring Type A polymerase. A further embodiment of the invention generally relates to a method wherein a modified Type A polymerase, e.g., in a primer extension, end-modification (e.g., terminal transferase, degradation, or polishing), or amplification reaction, may be selected from any species of the genus Meiothermus, Therrnotoga, or Thermomicrobium. Another embodiment of the invention generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the invention generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the invention generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the invention generally pertains to a method wherein a thermostable polymerase may be used to effect amplification of the target nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated.

The term “primer” refers to an oligonucleotide which binds to a specific region of a single-stranded template nucleic acid molecule and initiates nucleic acid synthesis via a polymerase-mediated enzymatic reaction. Typically, a primer comprises fewer than about 100 nucleotides and preferably comprises fewer than about 30 nucleotides. A target-specific primer specifically hybridizes to a target polynucleotide under hybridization conditions. Such hybridization conditions can include, but are not limited to, hybridization in isothermal amplification buffer (20 mM Tris-HCl, 10 mM (NH₄)₂SO₄), 50 mM KCl, 2 mM MgSO₄, 0.1% TWEEN® 20, pH 8.8 at 25° C.) at a temperature of about 40° C. to about 70° C. In addition to the target-binding region, a primer may have additional regions, typically at the 5′-portion. The additional region may include universal primer binding site or a barcode.

The term “sample” refers to any biological sample that comprises nucleic acid molecules, typically comprising DNA or RNA. Samples may be tissues, cells or extracts thereof, or may be purified samples of nucleic acid molecules. The term “sample” refers to any composition containing or presumed to contain target nucleic acid. Use of the term “sample” does not necessarily imply the presence of target sequence among nucleic acid molecules present in the sample. The sample can be a specimen of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). The sample can be collected from a non-human subject or from the environment.

The term “target” or “target nucleic acid” refer to the nucleic acid of interest in the sample. The sample may contain multiple targets as well as multiple copies of each target.

The term “universal primer” refers to a primer that can hybridize to a universal primer binding site. Universal primer binding sites can be natural or artificial sequences typically added to a target sequence in a non-target-specific manner.

Nucleic acid sequencing is rapidly expanding into clinical practice. The current sequencing technologies employ single molecule sequencing and allow detection of extremely rare targets. Among the clinical applications of nucleic acid sequencing is “liquid biopsy” e.g., detection and monitoring of malignant tumors using a blood sample instead of a traditional invasive biopsy. Tumor DNA is distinguished by the presence of mutations, including single nucleotide variations or small sequence variations as well as gene fusions. See Newman, A., et al., (2014) An ultrasensitive method for quantitating circulating tumor DNA with broad patient coverage, Nature Medicine doi:10.1038/nm.3519. Another clinical application is prenatal testing and prenatal diagnosis utilizing a sample of maternal blood containing small amounts of fetal DNA. Many more precision sequencing applications include infectious disease, molecular toxicology and other applications requiring accurate detection of rare nucleic acid sequences.

Many library preparation workflows exist for the separate preparation of DNA samples and RNA samples. When information is desired from both DNA and RNA, it is especially advantageous to have a combined workflow as described herein so a single source (e.g., a single patient specimen) could be used. This reduces the need for sample material as well as eliminates errors. This is especially advantageous for precious samples, such as clinical plasma or formalin fixed paraffin embedded tissue (FFPET) samples, forensic samples or historical or archival samples. Moreover, for these precious samples, there may not even be enough material to perform two separate isolations for DNA and RNA. With simultaneous analysis of DNA and RNA from a single source as described herein, the additional information gleaned from the second type of nucleic acid can be significant. For example, DNA holds the information about mutations, including single nucleotide variants (SNVs) and copy number variations (CNVs). In addition, the information derived from the DNA can be quantitative, i.e., reflect not only the type of mutation but also the mutation burden in the tumor sample. By contrast, RNA provides qualitative information about mutations as the varying expression levels obscure the mutation burden in the genome. At the same time, gene transcription amplifies the signal from a rare mutation event making it easier to detect. Analysis of RNA is especially useful for detecting gene fusions in the background of wild-type DNA sequences from both fusion partners.

Combined DNA/RNA workflows are known in the art U.S. application Ser. No. 15/611,507 filed on Jun. 1, 2017 (US Patent Application Publication No. 2018/0087108 A1). The present disclosure comprises the methods and reagents necessary to perform a combined DNA and RNA workflow without allowing the RNA-related steps to harm the DNA (e.g. through denaturation), and the current workflow has been optimized to minimize any negative impact on the DNA within the same tube. In some embodiments, the present invention further comprises a method of producing sequencing libraries from a mixture of DNA and RNA in a single tube. The method further enables tagging fragments originating from RNA which can then be separated from DNA during the sequence analysis.

The present invention comprises simultaneous isolation and sequencing of DNA and RNA target nucleic acids in a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). The sample may comprise whole blood or blood fractions where normal or tumor cells may be present. In some embodiments, the sample, especially a liquid sample may comprise cell-free material such as cell-free DNA or RNA including cell-free tumor DNA or tumor RNA. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain nucleic acids derived from the cells in the culture or from an infectious agent present in the culture. In some embodiments, the infectious agent is a bacterium, a protozoan, a virus or a mycoplasma.

Target nucleic acids are the nucleic acid of interest that may be present in the sample. Each target is characterized by its nucleic acid sequence. The present invention enables simultaneous detection of one or more RNA and DNA targets. In some embodiments, the DNA target nucleic acid is a gene or a gene fragment (including exons and introns) or an intergenic region, and the RNA target nucleic acid is a transcript or a portion of the transcript to which target-specific primers hybridize. In some embodiments, the target nucleic acid contains a locus of a genetic variant, e.g., a polymorphism, including a single nucleotide polymorphism or variant (SNP of SNV), or a genetic rearrangement resulting e.g., in a gene fusion. In some embodiments, the target nucleic acid comprises a biomarker, i.e., a gene whose variants are associated with a disease or condition. For example, the target nucleic acids can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Calif.) In other embodiments, the target nucleic acid is characteristic of a particular organism and aids in identification of the organism or a characteristic of the pathogenic organism such as drug sensitivity or drug resistance. In yet other embodiments, the target nucleic acid is a unique characteristic of a human subject, e.g., a combination of HLA or KIR sequences defining the subject's unique HLA or KIR genotype. In yet other embodiments, the target nucleic acid is a somatic sequence such as a rearranged immune sequence representing an immunoglobulin (including IgG, IgM and IgA immunoglobulin) or a T-cell receptor sequence (TCR). In yet another application, the target is a fetal sequence present in maternal blood, including a fetal sequence characteristic of a fetal disease or condition or a maternal condition related to pregnancy.

In some embodiments, the target nucleic acid is RNA (including mRNA, microRNA, viral RNA). In other embodiments, the target nucleic acid is DNA including cellular DNA or cell-free DNA (cfDNA) including circulating tumor DNA (ctDNA). The target nucleic acid may be present in a short or long form. Longer target nucleic acids may be fragmented. In some embodiments, the target nucleic acid is naturally fragmented, e.g., circulating cell-free DNA (cfDNA) or chemically degraded DNA such as the one founds in preserved samples.

In some embodiments, the invention comprises a step of nucleic acid isolation. Generally, any method of nucleic acid extraction that yields a mixture of isolated nucleic acids comprising DNA and RNA may be used. Genomic DNA and RNA may be extracted from tissues, cells, liquid biopsy samples (including blood or plasma samples) using solution-based or solid-phase based nucleic acid extraction techniques. Nucleic acid extraction can include detergent-based cell lysis, denaturation of nucleoproteins, and optionally removal of contaminants. Extraction of nucleic acids from preserved samples may further include a step of deparaffinization. Solution based nucleic acid extraction methods may comprise salting out methods or organic solvent or chaotrope methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Calif.) or AMPure beads (Beckman Coulter, Brea, Calif.)

A typical extraction method involves lysis of tissue material and cells present in the sample. Nucleic acids released from the lysed cells can be bound to a solid support (beads or particles) present in solution or in a column, or membrane where the nucleic acids may undergo one or more washing steps to remove contaminants including proteins, lipids and fragments thereof from the sample. Finally, the bound nucleic acids can be released from the solid support, column or membrane and stored in an appropriate buffer until ready for further processing. Because both DNA and RNA must be isolated, no nucleases may be used and care should be taken to inhibit any nuclease activity during the purification process. In some embodiments, longer nucleic acids such as genomic DNA can be sheared or fragmented into smaller genomic DNA fragments for example by sonication or enzymatic shearing.

In one embodiment, the invention comprises a method of simultaneously isolating DNA and RNA in a single tube and forming a library comprising targets derived from the isolated DNA and isolated RNA.

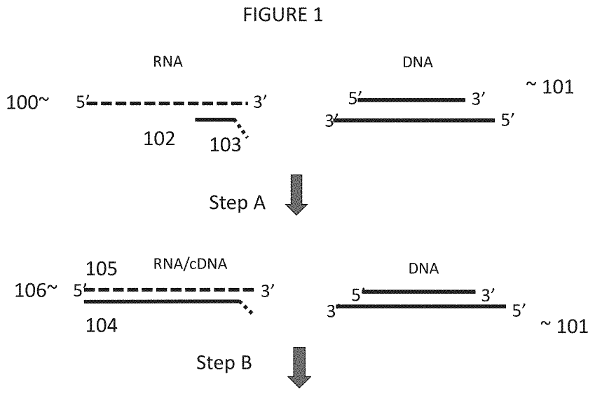

Referring to FIG. 1, the sample comprises RNA 100 and DNA 101. DNA may be partially single-stranded, i.e., having an overhang at one or both ends. The sample is contacted with a primer 102 binds to the RNA target. The primer comprises a tag 103 identifying the progeny of the RNA molecule 100 and distinguishing such progeny from the DNA molecule 101 and its progeny. The primer does not bind to the DNA target. In some embodiments, primer 102 is a gene specific primer. The primer may be prevented from binding to the DNA target by virtue of its sequence. In other embodiments, primer 102 is contacting the sample under conditions where DNA targets remain double stranded and inaccessible to the primer. In some embodiments, the sample is maintained at the temperature and salt conditions that allow for primer-RNA binding but do not favor DNA duplex denaturation. In some embodiments, the conditions comprise gentle heating in the presence of salt, e.g., 75 mM KCl+3 mM MgCl₂.

Referring further to FIG. 1, in step A, the sample is contacted with a DNA polymerase having reverse transcriptase activity. Extending primer 102 forms a primer extension product 104 in an RNA-DNA hybrid 106 consisting of the DNA strand 104 and RNA strand 105.

In Step B, the sample is contacted with RNase H which fragments the RNA strand 105 in the RNA-DNA hybrid 106. In some embodiments, it is advantageous to contact the hybrid 106 with a mild activity of RNase H to limit the degree of fragmentation of the RNA strand 105.

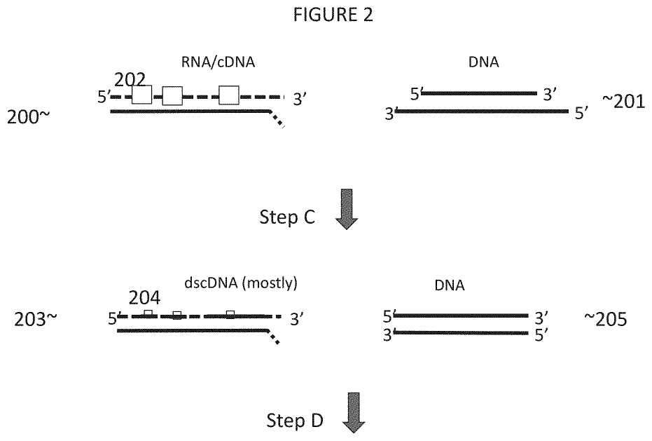

Referring to FIG. 2, the sample now contains RNA-DNA hybrid 200 with fragmented (partially degraded) RNA strand 202 and DNA 201. Notably, DNA 201 is identical to DNA 101 in FIG. 1. In Step C, the sample is contacted with DNA repair enzymes. In some embodiments, the DNA repair enzymes comprise a DNA polymerase which has 5′-3′ polymerase activity and 3′-5′ single stranded exonuclease activity, a polynucleotide kinase which adds a 5′ phosphate to the dsDNA molecule, and a DNA polymerase which adds a single dA base at the 3′ end of the dsDNA molecule. The end repair/A-tailing kits are available e.g., Kapa Library Preparation, kits including KAPA Hyper Prep and KAPA HyperPlus (Kapa Biosystems, Wilmington, Mass.). The repair enzymes convert the partially degraded RNA strand 202 into a cDNA strand 204 in a mostly double stranded cDNA molecule 203. The same repair enzymes create blunt ends in DNA 201 to form blunt-ended DNA 205.

In some embodiments, Step B and Step C occur simultaneously wherein the sample is contacted with RNaseH and repair enzymes at the same time.

Referring further to FIG. 2, in Step D, the DNA polishing and A-tailing enzymes create fully double stranded DNA with blunt ends and add a single A (deoxyriboadenosine nucleoside) to each of the 3′-ends.

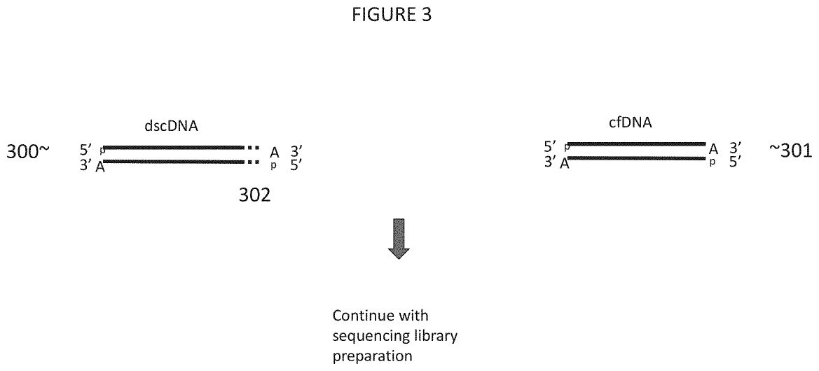

Referring to FIG. 3, after step D, the sample now contains fully double stranded and A-tailed cDNA 300 and fully double stranded and A-tailed DNA 301. Notably, DNA 300 is distinguishable from DNA 301 by the presence of barcode 302 identifying the progeny of RNA from the progeny of DNA. The cDNA 300 and DNA 301 are ready for further steps in the sequencing workflow such as, for example adaptor ligation, amplification or target capture or any combination of the foregoing in any order desired by the user.

In some embodiments, the input DNA or input RNA require fragmentation prior to Step A (FIG. 1). In such embodiments, RNA may be fragmented by a combination of heat and metal ions, e.g., magnesium. In some embodiments, the sample is heated to 85°−94° C. for 1-6 minutes in the presence of magnesium. (KAPA RNA HyperPrep Kit, KAPA Biosystems, Wilmington, Mass.). DNA can be fragmented by physical means, e.g., sonication, using available instruments (Covaris, Woburn. Mass.) or enzymatic means (KAPA Fragmentase Kit, KAPA Biosystems).

In some embodiments, DNA is damaged and requires pre-treatment prior to Step A (FIG. 1). In some embodiments, DNA is partially damaged DNA from preserved samples, e.g., formalin-fixed paraffin embedded (FFPET) samples. In some embodiments, the damaged DNA is treated with uracil N-DNA glycosylase (UNG/UDG) and/or 8-oxoguanine DNA glycosylase.

In some embodiments, the invention utilizes target-specific primers. A target specific primer comprises at least a portion that is complementary to the target. If additional sequences are present, such as the barcode 103 (FIG. 1), they are typically located in the 5′-portion of the primer. The target may be a gene sequence (coding or non-coding) or a regulatory sequence present in RNA 100 (FIG. 1) such as an enhancer or a promoter.

In some embodiments, the invention comprises a step of adaptor ligation. The adaptor may be ligated to the ends of a double stranded DNA molecule formed as described herein. Adaptors of various shapes and functions are known in the art, see e.g., U.S. Pat. Nos. 8,153,375, 8,822,150 and International Application Ser. No. PCT/EP2019/055015 “Generation of double-stranded DNA templates for single-molecule sequencing” (published as WO 2019/166565).

The adaptor may be double-stranded, partially single stranded or single stranded. In some embodiments, a Y-shaped, a hairpin adaptor or a stem-loop adaptor is used wherein the double-stranded portion of the adaptor is ligated to the double stranded nucleic acid formed as described herein.

In some embodiments, the adaptor molecules are in vitro synthesized artificial sequences. In other embodiments, the adaptor molecules are in vitro synthesized naturally-occurring sequences. In yet other embodiments, the adaptor molecules are isolated naturally occurring molecules or isolated non naturally-occurring molecules.

In some embodiments, the adaptor comprises one or more barcodes. A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

The adaptor further comprises a primer binding site for at least one universal primer.

The double-stranded or partially double-stranded adaptor oligonucleotide can have overhangs or blunt ends. In some embodiments, the double-stranded DNA formed by the method described herein comprises blunt ends to which a blunt-end ligation can be applied to ligate a blunt-ended adaptor. In other embodiments, the blunt ended DNA undergoes A-tailing where a single A nucleotide is added to the blunt ends to match an adaptor designed to have a single T nucleotide extending from the blunt end to facilitate ligation between the DNA and the adaptor. Commercially available kits for performing adaptor ligation include AVENIO ctDNA Library Prep Kit or KAPA HyperPrep and HyperPlus kits (Roche Sequencing Solutions, Pleasanton, Calif.). In some embodiments, the adaptor ligated (adapted) DNA may be separated from excess adaptors and unligated DNA.

Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient's sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient's blood in order to detect and monitor cancer without a biopsy (Newman, A., et al., (2014) An ultrasensitive method for quantitating circulating tumor DNA with broad patient coverage, Nature Medicine doi:10.1038/nm.3519).

Unique molecular barcodes can also be used for sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the invention comprises an amplification step. The double-stranded DNA fragments prepared by the method described herein or optionally adapted nucleic acids can be amplified prior to sequencing This step can involve linear or exponential amplification, e.g., PCR. Amplification may be isothermal or involve thermocycling. In some embodiments, the amplification is exponential and involves PCR. In some embodiments, universal primers are used, i.e., a pair of primers that hybridizes to a universal primer binding site in the adaptor present on all target sequences in the sample. All molecules in the library having the same adaptor containing a universal primer binding site can be amplified with the same set of primers. The number of amplification cycles where universal primers are used can be low but also can be 10, 20 or as high as about 30 or more cycles, depending on the amount of product needed for the subsequent steps. Because PCR with universal primers has reduced sequence bias, the number of amplification cycles need not be limited to avoid amplification bias.

In some embodiments, the method comprises only one round of amplifying adapted nucleic acids prior to sequencing. In other embodiments, the method comprises additional rounds of amplification, e.g., after enrichment or capture as described herein.

In some embodiments, the invention further comprises a step of target enrichment. In some embodiments, the method utilizes a pool of oligonucleotide probes (e.g., capture probes). The enrichment can be by subtraction in which case, capture probes are complementary to an abundant undesired sequences including ribosomal RNA (rRNA) or abundantly expressed genes (e.g., globin). In the case of subtraction, the undesired sequences are captured by the capture probes and removed from the solution of target nucleic acids and discarded, e.g., utilizing capture probes with a binding moiety that can be captured on solid support. In other embodiments, the enrichment can be capture in which case, capture probes are complementary to one or more target sequences. In this case the target sequences are captured by the capture probes and removed from the solution, e.g., utilizing capture probes with a binding moiety that can be captured on solid support, while the remained of the solution is discarded.

For enrichment, the capture probes may be free in solution or fixed to solid support. The probes may also comprise a binding moiety (e.g., biotin) and be capable of being captured on solid support (e.g., avidin or streptavidin containing support material).

In some embodiments, the invention comprises intermediate purification steps. embodiments, the unused primers and adaptors are removed, e.g., by a size selection method selected from gel electrophoresis, affinity chromatography and size exclusion chromatography. In some embodiments, size selection can be performed using Solid Phase Reversible Immobilization (SPRI) technology from Beckman Coulter (Brea, Calif.).

The adapted nucleic acids described by the method disclosed herein or amplicons thereof can be subjected to nucleic acid sequencing. Sequencing can be performed by any method known in the art. Especially advantageous is the high-throughput single molecule sequencing. Examples of such technologies include the Illumina HiSeq platform (Illumina, San Diego, Calif.), Ion Torrent platform (Life Technologies, Grand Island, N.Y.), Pacific BioSciences platform utilizing the SMRT (Pacific Biosciences, Menlo Park, Calif.) or a platform utilizing nanopore technology such as those manufactured by Oxford Nanopore Technologies (Oxford, UK) or Roche Sequencing Solutions (Santa Clara, Calif.) and any other presently existing or future DNA sequencing technology that does or does not involve sequencing by synthesis. The sequencing step may utilize platform-specific sequencing primers. Binding sites for these primers may be introduced in 5′-portions of the amplification primers used in the amplification step. If no primer sites are present in the library of barcoded molecules, an additional short amplification step introducing such binding sites may be performed.

In some embodiments, the sequencing step involves sequence analysis. In some embodiments, the analysis includes a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the invention is a library of target nucleic acids derived from RNA and DNA targets as disclosed herein. The library formed by a method described herein comprises double-stranded DNA molecules where the molecules originating from RNA targets present in the original sample are characterized by a barcode absent from the molecules originating from RNA targets present in the original sample. The library molecules may further comprise adaptors added after completion of the method steps described herein in FIGS. 1-3.

### EXAMPLES

### Example 1. Simultaneously Preparing Sequencing-Ready DNA and RNA from a Cell Line Sample

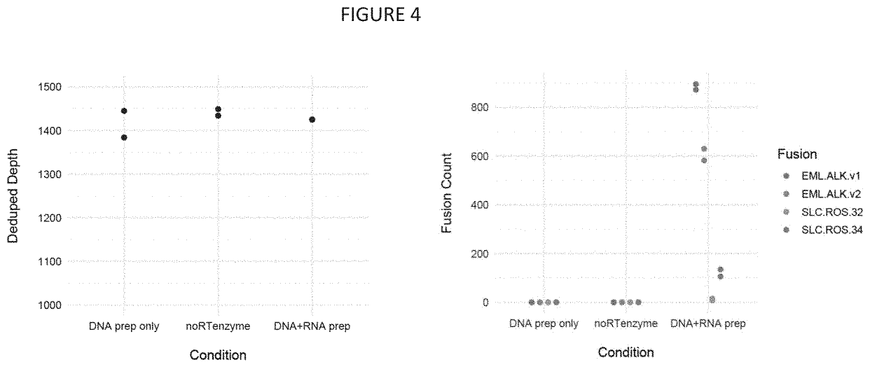

To generate the data described in FIG. 4, an optimized DNA/RNA workflow was used on cell line DNA/cell line RNA blends with cell lines containing known fusions (EML4-ALK and SLC34A2-ROS1 fusions). Gene specific primers were used to target the relevant exons in ALK and ROS1 during reverse transcription. As noted above, reverse transcription was performed in such a way that DNA maintained its ability to be library prepped (i.e. stayed in duplex and was relatively undamaged). After reverse transcription, RNAseH treatment was performed, the sample was taken into the End Repair/A-tailing step of the AVENIO Tumor Tissue Analysis kit workflow (Roche Sequencing Solutions, Pleasanton, Calif.), and the rest of the workflow was performed using the AVENIO tumor tissue protocol. The goal of this experiment was to determine if RNA fusions could be detected while maintaining the expected depth from the DNA within the sample. As such, this method was compared to a “DNA prep only” method, using only DNA as input and following the AVENIO Tumor Tissue protocol exactly, and a “no RT enzyme” condition to ensure that none of the detected fusions were from RNA. FIG. 4 shows that (1) fusions are detected only in the combined DNA/RNA prep, and all expected fusions are detected in that prep, and (2) the DNA/RNA prep does not lose depth in the sample relative to the optimized DNA only prep.

1. A method of preparing a mixture of RNA and DNA targets for sequencing the method comprising:
a) providing a sample comprising RNA and DNA targets;
b) contacting the sample with a target-specific primer under conditions that do not allow DNA denaturation;
c) extending the target-specific primer hybridized to at least one RNA target with a nucleic acid polymerase having reverse transcriptase activity to form a cDNA strand;
d) contacting the sample with an RNaseH activity and a nucleic acid end repair activity to form a mixture of double-stranded DNA and double-stranded cDNA.

2. The method of claim 1, wherein the target-specific primer comprises a barcode.

3. The method of claim 2, wherein the barcode distinguishes cDNA molecules from DNA molecules in the mixture of double-stranded DNA and double-stranded cDNA.

4. The method of claim 1, wherein the nucleic acid end repair activity consists of a mixture of a DNA polymerase, an exonuclease and a polynucleotide kinase.

5. The method of claim 1, further comprising a preliminary step of fragmenting the RNA and DNA targets.

6. The method of claim 1, further comprising contacting the mixture of double-stranded DNA and double-stranded cDNA with an adaptor to form adapted DNA.

7. The method of claim 6, wherein the adaptor comprises one or more barcodes.

8. The method of claim 7, wherein the barcode is selected from a unique molecular identifier (UID) and a sample identifier (SID).

9. The method of claim 6, further comprising a step of amplifying and optionally sequencing the adapted DNA.

10. A library of nucleic acids formed by a method comprising providing a sample comprising RNA and DNA targets;
c) contacting the sample with a target-specific primer under conditions that do not allow DNA denaturation;
c) extending the target-specific primer hybridized to at least one RNA target with a nucleic acid polymerase having reverse transcriptase activity to form a cDNA strand; and
d) contacting the sample with an RNaseH activity and a nucleic acid end repair activity to form a mixture of double-stranded DNA and double-stranded cDNA.

11. The library of claim 10, wherein the DNA in mixture of double-stranded DNA and double-stranded cDNA further comprises adaptors.

12. The library of claim 10, wherein the target-specific primer comprises a barcode and the double-stranded cDNA is distinguishable from the double-stranded DNA by the presence of the barcode.

13. A kit for preparing a mixture of RNA and DNA targets for sequencing by the method according to claim 1, the kit comprising:
d) one or more target-specific primers having a barcode;
e) a nucleic acid polymerase having reverse transcriptase activity;
f) an RNaseH;
g) a DNA polymerase having a 3′-5-exonuclease activity;
h) a polynucleotide kinase.

14. The kit of claim 13, further comprising an adaptor and a DNA ligase.

15. The kit of claim 14, wherein the adaptor comprises one or more molecular barcodes and a universal primer binding site.
